(19) United States
(12) Patent Application Publication
(10) Pub. No.: US 2010/0129301 A1
(43) Pub. Date: May 27, 2010

(54) COSMETIC COMPOSITIONS

(76) Inventors: Shuhei Tanaka, Kobe (JP); Naohisa Yoshimi, Nishinomiya (JP)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/624,236

(22) Filed: Nov. 23, 2009

Related U.S. Application Data
(60) Provisional application No. 61/117,264, filed on Nov. 24, 2008.

(51) Int. Cl. A61K 8/40; A61Q 17/04

(52) U.S. Cl. 424/59

(57) ABSTRACT

A cosmetic composition comprises a) from about 0.1% to about 15% of an emulsifying crosslinked siloxane elastomer; b) from about 0.1% to about 10% of a noncrosslinked silicone emulsifier having a polyglycerin unit; c) from about 1% to about 50% of a solvent for the emulsifying crosslinked siloxane elastomer; d) from about 0.05% to about 10% of an amphiphilic active; and e) water.

### CROSS REFERENCE TO RELATED APPLICATION

This application claims the benefit of U.S. Provisional Application No. 61/117,264, filed on Nov. 24, 2008.

### FIELD OF THE INVENTION

The present invention relates to skin care compositions comprising a noncrosslinked silicone emulsifier having a polyglycerin unit and skin care actives.

### BACKGROUND OF THE INVENTION

Mammalian keratinous tissue, particularly human skin, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage. Typical skin damage in aging or damaged skin include fine lines, wrinkling, hyperpigmentation, sallowness, sagging, dark under-eye circles, puffy eyes, enlarged pores, diminished rate of turnover, and abnormal desquamation or exfoliation. Additional damage incurred as a result of both external and internal factors includes visible dead skin (i.e., flaking, scaling, dryness, roughness).

Currently, there are a number of personal care products that are available to consumers, which are directed toward improving the health and physical appearance of keratinous tissues such as the skin, hair, and nails. The majority of these products are directed to delaying, minimizing or even eliminating skin wrinkling and other histological changes typically associated with the aging of skin or environmental damage to human skin. Consumers prefer topically applied products since they are not only effective, but also safe and pleasant to use. Upon application of such topical products, they should not run or be sticky.

Some of effective skin care actives, usually salt form materials or amphiphilic materials are know to be difficult to formulate especially in an aqueous environment since these materials would lessen the thickening ability of a composition, and finally cause significant viscosity drop or phase separation in the composition. Adding a thickener or increasing the level of a thickener in the composition may help the formulation robustness but usually accompany in inferior skin feel and decreased perception of absorption into the skin.

Based on the foregoing, there is a continuing need to formulate skin care compositions that can provide stable delivery of skin actives without deteriorating product stability and sensory acceptance.

None of the existing art provides all of the advantages and benefits of the present invention.

### SUMMARY OF THE INVENTION

The present invention relates to a cosmetic composition comprising a) from about 0.1% to about 15% of an emulsifying crosslinked siloxane elastomer; b) from about 0.1% to about 3% of a noncrosslinked silicone emulsifier having a polyglycerin unit; c) from about 1% to about 50% of a solvent for the emulsifying crosslinked siloxane elastomer; d) from about 0.05% to about 10% of an amphiphilic active; and e) water.

The present invention also relates to methods of using such compositions to regulate the condition of skin, said method comprising applying to the skin of a human in need of treatment.

These and other features, aspects, and advantages of the present invention will become evident to those skilled in the art from a reading of the present disclosure.

### DETAILED DESCRIPTION OF THE INVENTION

While the specification concludes with the claims particularly pointing and distinctly claiming the invention, it is believed that the present invention will be better understood from the following description.

As used herein, the “skin care products” are those used to treat or care for, or somehow moisturize, improve, or clean the skin. Products contemplated by the phrase “skin care products” comprise, but are not limited to moisturizers, personal cleansing products, occlusive drug delivery patches, nail polish, powders, wipes, hair conditioners, skin treatment emulsions, shaving creams and the like.

The compositions of the present invention can include, consist essentially of, or consist of, the components of the present invention as well as other ingredients described herein.

The term “ambient conditions” as used herein refers to surrounding conditions under about one atmosphere of pressure, at about 50% relative humidity, and at about 25° C. unless otherwise specified.

The term “an amphiphilic active” as used herein refers to a component having both a hydrophilic and hydrophobic moieties, and is a negatively or positively charged form in an aqueous environment.

The term “keratinous tissue” as used herein, refers to keratin-containing layers disposed as the outermost protective covering of mammals (e.g., humans, dogs, cats, etc.) which includes, but is not limited to, skin, lips, hair, toenails, fingernails, cuticles, hooves, etc.

The term “safe and effective amount” as used herein, refers to an amount of a compound or composition sufficient to significantly induce a positive benefit, preferably a positive keratinous tissue appearance or feel benefit, or positive hair appearance or feel benefit, including independently or in combinations the benefits disclosed herein, but low enough to avoid serious side effects, i.e., to provide a reasonable benefit to risk ratio, within the scope of sound judgment of the skilled artisan.

The term “regulating skin condition” as used herein, refers to improving skin appearance and/or feel, for example, by providing a benefit, such as a smoother appearance and/or feel. The benefit may be a chronic benefit and may include one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

All percentages, parts and ratios are based upon the total weight of the skin care compositions of the present invention, unless otherwise specified. All such weights as they pertain to listed ingredients are based on the active level and, therefore, do not include carriers or by-products that may be included in commercially available materials, unless otherwise specified.

The compositions of the present invention are useful for regulating the condition of skin and especially for regulating keratinous tissue condition.

The compositions of the present invention provide additional benefits, including stability, absence of significant (consumer-unacceptable) skin irritation and good aesthetics.

In some embodiments the compositions herein are in the form of water-in-oil emulsion and contain an emulsifying crosslinked siloxane elastomer, a polyglycerin-modified noncrosslinked silicone emulsifier, a solvent for the emulsifying crosslinked siloxane elastomer, an amphiphilic active, and water.

The compositions of the present invention optionally contain a non-emulsifying crosslinked siloxane elastomer, an additional emulsifier, a sunscreen agent and/or additional oil. The compositions also preferably contain one or more skin care actives in addition to the amphiphilic active. The nature of the actives and other ingredients depending on their nature, can be introduced into the aqueous phase or into one of the oil phases of the present emulsion.

The compositions herein may also include a wide variety of other ingredients. The compositions of the present invention are described in detail hereinafter.

### Emulsifying Crosslinked Siloxane Elastomers

The compositions of the present invention comprise an emulsifying crosslinked siloxane elastomer. The emulsifying crosslinked siloxane elastomer is present in the compositions of the present invention at concentrations of from about 0.1% to about 15%, preferably from about 0.25% to about 10%, most preferably from about 0.5% to about 5% by weight. The indicated percentages are understood to refer to amount of dry elastomer, as opposed to the total amount of elastomers and solvent, used for example for storage or shipping.

The term “emulsifying crosslinked siloxane elastomer,” as used herein, means crosslinked organopolysiloxane elastomer having at least one polyoxyalkylene (e.g., polyoxyethylene or polyoxypropylene) or polyglycerin moiety.

Emulsifying crosslinked siloxane elastomers in the present invention include those described in U.S. Pat. Nos. 5,412,002; 5,837,793 and 5,811,487. None-limiting examples of useful emulsifying crosslinked siloxane elastomers are 1) polyoxyalkylene-modified elastomers formed from divinyl compounds, particularly siloxane polymers with at least two free vinyl groups, reacting with Si—H linkages on a polysiloxane backbone. Such emulsifying crosslinked siloxane elastomers include KSG-210, KSG-240, KSG-310, KSG-320 and KSG-330 available by Shin-Etsu. Another preferred emulsifying crosslinked siloxane elastomers are siloxane polymers crosslinked with polyglycerin unit such as KSG-710, KSG-810, KSG-820, KSG-830 and KSG840 available from Shin-Etsu.

### Non-Crosslinked Silicone Emulsifier Having a Polyglycerin Unit

The compositions of the present invention comprise a non-crosslinked silicon emulsifier having a polyglycerin unit. The non-crosslinked silicon emulsifier having a polyglycerin unit is present in the compositions of the present invention at concentrations of from about 0.1 to about 10.0%, preferably from about 0.1 to about 5.0%, most preferably from about 0.3% to about 3.0% by weight.

Silicone emulsifiers having a polyglycerin unit include polydimethyl siloxanes which have been modified to include polyglycerin side chain such as KF-6104 and KF-6100 available from ShinEtsu. Other examples of silicone emulsifiers having a polyglycerin unit include silicone emulsifiers which have been modified to include an alkyl chain as well as a polyglycerin unit such as KF-6105 available from ShinEtsu.

### Solvent for an Emulsifying Crosslinked Siloxane Elastomer

The compositions of the present invention comprise a solvent for an emulsifying crosslinked siloxane elastomer. Concentrations of the solvent in the cosmetic compositions of the present invention will vary primarily with the type and amount of solvent and the emulsifying crosslinked siloxane elastomer employed. Concentrations of the solvent may be from about 1% to about 50%, preferably from about 5% to about 50%, more preferably from about 10% to about 40%, by weight of the composition.

The solvent, when combined with the emulsifying crosslinked siloxane elastomer particles, serves to suspend and swell the elastomer particles to provide an elastic, gel-like network or matrix. The solvent is not limited to those provided as a component of commercially available emulsifying crosslinked siloxane elastomer dispersion.

The solvent for the emulsifying cross-linked siloxane elastomer is liquid under ambient conditions, and in one embodiment has a low viscosity to provide for improved spreading on the skin.

The solvent for the emulsifying crosslinked siloxane elastomer may comprise one or more liquid carriers suitable for topical application to human skin. These liquid carriers may be organic, silicone-containing or fluorine-containing, volatile or non-volatile, polar or non-polar, provided that the liquid carrier forms a solution or other homogenous liquid or liquid dispersion with the selected emulsifying crosslinked siloxane elastomer at the selected siloxane elastomer concentration at a temperature of from about 28° C. to about 250° C., preferably from about 28° C. to about 100° C., preferably from about 28° C. to about 78° C. The solvent for the emulsifying crosslinked siloxane elastomer preferably has a solubility parameter of from about 3 to about 13 (cal/cm³)^{0.5}, more preferably from about 5 to about 11 (cal/cm³)^{0.5}, most preferably from about 5 to about 9 (cal/cm³)^{0.5}. Solubility parameters for the liquid carriers or other materials, and means for determining such parameters, are well known in the chemical arts. A description of solubility parameters and means for determining them are described by C. D. Vaughan, “Solubility Effects in Product, Package, Penetration and Preservation” 103 Cosmetics and Toiletries 47-69, October 1988; and C. D. Vaughan, “Using Solubility Parameters in Cosmetics Formulation”, 36 J. Soc. Cosmetic Chemists 319-333, September/October, 1988.

The solvent preferably includes volatile, non-polar oils; non-volatile, relatively polar oils; non-volatile, non-polar oils; and non-volatile paraffinic hydrocarbon oils; each discussed more fully hereinafter. The term “non-volatile” as used herein refers to materials that exhibit a vapor pressure of no more than about 0.2 mm Hg at 25° C. at one atmosphere and/or to materials that have a boiling point at one atmosphere of at least about 300° C. The term “volatile” as used herein refers to all materials that are not “non-volatile” as previously defined herein. The phrase “relatively polar” as used herein means more polar than another material in terms of solubility parameter; i.e., the higher the solubility parameter the more polar the liquid. The term “non-polar” typically means that the material has a solubility parameter below about 6.5 (cal/cm³)^{0.5}.

Non-limiting examples of suitable non-polar, volatile oil are disclosed in U.S. Pat. No. 4,781,917 issued to Luebbe et al. and include polydecanes such as isododecane and isodecane (e.g., Permethyl-99A, available from Presperse™ Inc.) and C7-C15 isoparaffins (e.g. the Isopar Series, from Exxon™ Chemicals); silicone oil of cyclomethicones of varying viscosities, e.g., Dow Corning™ 200, Dow Corning™ 244, Dow Corning™ 245, Dow Corning™ 344, and Dow Corning™ 345, Silicone Fluids, commercially available from G.E. Silicones, (e.g. SF-1204, SF-1202, GE 7207 and GE 7158); and SWS-03314 (commercially available from SWS Silicones™ Corp.).

Polar, non-volatile oils useful in the present invention include, but are not limited to, silicone oils; hydrocarbon oils; and mixtures thereof. In one embodiment, the polar, non-volatile oil is selected from the group consisting of propoxylated ethers of C14-C18 fatty alcohols having a degree of propoxylation below about 50, esters of C2-C8 alcohols and C12-C26 carboxylic acids (e.g. ethyl myristate, isopropyl palmitate), esters of C12-C26 alcohols and benzoic acid (e.g. Finsolv™ TN supplied by Finetex™), diesters of C2-C8 alcohols and adipic, sebacic, and phthalic acids (e.g., diisopropyl sebacate, diisopropyl adipate, di-n-butyl phthalate), polyhydric alcohol esters of C6-C26 carboxylic acids (e.g., propylene glycol dicaprate/dicaprylate, propylene glycol isostearate); and mixtures thereof.

Examples of suitable non-volatile, non-polar oils include, but are not limited to, non-volatile polysiloxanes, paraffinic hydrocarbon oils, and mixtures thereof. The polysiloxanes useful in the present invention selected from the group consisting of polyalkylsiloxanes, polyarylsiloxanes, polyalkylarylsiloxanes, poly-ethersiloxane copolymers, and mixtures thereof. Examples of useful oils include Viscasil™ series (General Electric); Dow Corning 200 series (Dow Corning Corp.); SF 1075 methyl-phenyl fluid (General Electric) and 556 Cosmetic Grade Fluid (Dow Corning Corp.).

Non-volatile paraffinic hydrocarbon oils useful in the present invention are described in U.S. Pat. No. 5,019,375 issued to Tanner et al. and in 2003/0049212A1, and include mineral oils and branched-chain hydrocarbons such as Permethyl™ 102A, 103A and 104A (Permethyl Corporation); and Ethylflo™ 364 (Ethyl Corp.).

Additional solvents useful herein are described in U.S. Pat. No. 5,750,096 to Gerald J. Guskey et al., issued May 12, 1998.

### Amphiphilic Actives

The compositions according to the present invention comprise an amphiphilic active from 0.01% to 10%, preferably from about 0.05% to 5% and more preferably from 0.1% to 2% by weight of the composition. The amphiphilic active may be either positively or negatively charged in an aqueous environment.

Examples of the amphiphilic active in the present invention include, but are not limited to undecenoyl phenylalanine available from Seppic as Sepiwhite MSH, cetylpyridinium chloride, glycyrrhizate salts including ammonium glycyrrhizate, dipotassium glycyrrhizate, disodium glycyrrhizate, tripotassium glycyrrhizate, trisodium glycyrrhizate, sodium dilauramidoglutamide lysine, and olive oil derivatives such as sodium PEG-7 olive oil carboxylate available from B&T SRL as Olivem 400, Olivem 450 or Olivem 460.

### Water

The cosmetic compositions of the present invention comprise water preferably from 10% to 90%, more preferably from about 30% to 80% and more preferably from 40% to 60% by weight of the composition.

### Optional Components

### Non-Emulsifying Crosslinked Siloxane Elastomers

The compositions of the present invention can optionally contain non-emulsifying crosslinked siloxane elastomers. The term “non-emulsifying crosslinked siloxane elastomers,” as used herein, defines crosslinked organopolysiloxane elastomer from which polyoxyalkylene units or polyglycerin units are absent.

Non-limiting examples of non-emulsifying crosslinked siloxane elastomers used herein include dimethicone/vinyl dimethicone crosspolymers, supplied by a variety of suppliers including Dow Corning™ (DC 9040 and DC 9041), General Electric™ (SFE 839), Shin-Etsu™ (KSG-15, 16, 18 [dimethicone/phenyl vinyl dimethicone crosspolymer]), and Grant Industries (GRANSIL™ line of elastomers). Cross-linked siloxane elastomers useful in the present invention and processes for making them are further described in U.S. Pat. No. 4,970,252 to Sakuta, et al.; U.S. Pat. No. 5,760,116 to Kilgour, et al.; and U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-018708, assigned to Pola Kasei Kogyo KK. In addition, suitable organopolysiloxane elastomer powders include vinyl dimethicone/methicone silesquioxane crosspolymers such as KSP-100, KSP-101, KSP-102, KSP-103, KSP-104, KSP-105 (Shin-Etsu™); hybrid silicone powders comprising a fluoroalkyl group, such as KSP-200 (Shin-Etsu™); and hybrid silicone powders comprising a phenyl group, such as KSP-300 (Shin-Etsu™) and DC-9506 (Dow Corning™).

In some embodiments, the composition may contain from about 0.1 to about 15%, preferably from about 0.1 to about 5%, most preferably from about 0.1 to about 2% of a non-emulsifying crosslinked siloxane elastomer by weight of the composition.

### Additional Emulsifiers

The compositions of the present invention can optionally contain an additional emulsifier. In some embodiments, the composition may contain from about 0.01% to about 5%, preferably from 0.01% to about 3% additional emulsifier, more preferably from about 0.1% to about 3% of an additional emulsifier by weight of the composition. The additional emulsifier if present may help disperse and suspend a water phase within an oil phase.

Known or conventional emulsifying agents can be used in the composition, provided that the selected emulsifying agent is chemically and physically compatible with components of the compositions of the present invention, and provides the desired dispersion characteristics. Suitable emulsifiers are disclosed in, for example, U.S. Pat. No. 3,755,560, issued Aug. 28, 1973, Dickert et al.; U.S. Pat. No. 4,421,769, issued Dec. 20, 1983, Dixon et al.; and McCutcheon's Detergents and Emulsifiers, North American Edition, pages 317-324 (1986). Non-limiting examples of nonionic emulsifiers are alkoxylated compounds based on C10-C22 fatty alcohols and acids, and sorbitan. These materials are available, for instance, from the Shell Chemical Company under the Neodol trademark, Copolymers of polyoxypropylene-polyoxyethylene, sold by the BASF Corporation under the Pluronic trademark, are sometimes also useful. Alkyl polyglycosides available from the Henkel Corporation may also be utilized for purposes of this invention. Anionic type emulsifiers or surfactants include fatty acid soaps, sodium lauryl sulphate, sodium lauryl ether sulphate, alkyl benzene sulphonate, mono- and di-alkyl acid phosphates and sodium fatty acyl isethionate. Amphoteric emulsifiers or surfactants include such materials as dialkylamine oxide and various types of betaines (such as cocamidopiopyl betaine).

In one embodiment, the additional emulsifier is a silicone emulsifier. A wide variety of silicone emulsifiers is useful herein. These silicone emulsifiers are typically organically modified siloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C2-C30 pendant side chains. Still other useful dimethicone copolyols include materials having various cationic, anionic, amphoteric, and zwitterionic pendant moieties.

### Oils

The compositions of the present invention may contain at least one other oil which is not a solvent for the emulsifying siloxane elastomer. In the present invention, wide range of oils from polar oils to non polar oils can be used as other oil for the internal and/or external oil phase component. Other oils in the present invention include, but are not limited to, hydrocarbon oils and waxes, fatty alcohol and fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, salts, isomers and derivatives thereof, and combinations thereof.

Non-limiting examples of hydrocarbon oils and waxes suitable for use herein include, but are not limited to, petrolatum, mineral oil, micro-crystalline waxes, polyalkenes, paraffins, isoparaffin, branched-chain light paraffin, polyethylene, squalane, perhydrosqualene, and ester oils such as isopropyl myristate, cetyl isoocatanoate, and glyceryl trioctanoate, and combinations thereof.

### Skin Care Actives

The compositions of the present invention may include at least one skin care active. Without being bound by theory, it is believed the present compositions provide versatility in formulating a variety of actives.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

Vitamin B₃ Compounds

Vitamin B₃ compounds such as niacinamide are a preferred skin care active for use herein. The present invention preferably includes from about 0.1% to about 30%, more preferably from about 1% to about 20%, even more preferably from about 2% to about 10% of a vitamin B₃ compound.

As used herein, “vitamin B₃ compound” means a compound having the formula:

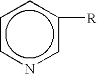

wherein R is —CONH₂ (i.e., niacinamide), —COOH (i.e., nicotinic acid) or —CH₂OH (i.e., nicotinyl alcohol); derivatives thereof; and salts of any of the foregoing. Exemplary derivatives of the foregoing vitamin B₃ compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B₃ compounds, azelaic acid, butyl hydroxy anisole, gallic acid and its derivatives, hydroquinoine, kojic acid, arbutin, mulberry extract, tetrahydrocurcumin, and mixtures thereof. Use of combinations of whitening agents is also believed to be advantageous in that they may provide whitening benefit through different mechanisms.

When used, the compositions preferably contain from about 0.1% to about 10%, more preferably from about 0.2% to about 5%, by weight of the composition, of a whitening agent.

Ascorbic acid compounds are useful whitening agents, and include compounds having the formula (I):

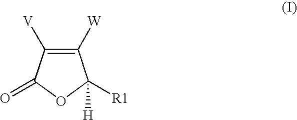

wherein V and W are independently —H or —OH; R¹ is —CH(OH)—CH₂OH; salts thereof; and derivatives thereof. Preferably, the ascorbic acid compound useful herein is an ascorbic acid salt or derivative thereof, such as the non-toxic alkali metal, alkaline earth metal and ammonium salts commonly known by those skilled in the art including, but not limited to, the sodium, potassium, lithium, calcium, magnesium, barium, ammonium and protamine salts which are prepared by methods well known in the art. Ascorbyl glucoside is a preferably derivative.

Peptides

Peptides, including but not limited to, di-, tri-, tetra-, and pentapeptides and derivatives thereof, may be included in the compositions of the present invention in amounts that are safe and effective. As used herein, “peptides” refers to both the naturally occurring peptides and synthesized peptides. Also useful herein are naturally occurring and commercially available compositions that contain peptides.

When included in the present compositions, peptides are preferably included in amounts of from about 1×10⁻⁶% to about 10%, more preferably from about 1×10⁻⁶% to about 0.1%, even more preferably from about 1×10⁻⁵% to about 0.01%, by weight of the composition.

Sugar Amines

The compositions of the present invention may include a safe and effective amount of a sugar amine, which are also known as amino sugars. As used herein, “sugar amine” refers to an amine derivative of a six-carbon sugar. Examples of sugar amines that are useful herein include glucosamine, N-acetyl glucosamine, mannosamine, N-acetyl mannosamine, galactosamine, N-acetyl galactosamine. Preferred for use herein is glucosamine. Additionally, combinations of two or more sugar amines may be used.

When included in the present compositions, a sugar amine is preferably included in amounts of from about 0.001% to about 20%, more preferably from about 1% to about 10%, even more preferably from about 2% to about 5%, by weight of the composition.

### Skin Conditioning Agent

Optionally, the composition of the present invention can further comprise a skin conditioning agent. These agents may be selected from humectants, exfoliants or emollients. The amount of skin-condition agent may range from about 1% to about 60%, preferably from about 2% to about 50%, more preferably from about 5% to about 40%, by weight of the composition.

Humectants are polyhydric alcohols intended for moisturizing, reducing scaling and stimulating removal of built-up scale from the skin. Typical polyhydric alcohols include polyalkylene glycols and more preferably alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. Most preferably the humectant is glycerin.

Exfoliants according to the present invention may be selected from C2-C30 alpha-hydroxycarboxylic acids, beta-hydroxycarboxylic acids and salts of these acids. Most preferred are glycolic, lactic and salicylic acids and their ammonium salts.

When the conditioning agent is an emollient it may be selected from hydrocarbons, fatty acids, fatty alcohols and esters.

### Oil-Soluble Compounds

The term “oil-soluble”, as used herein, means when calculating inorganic/organic balance, organic portion is equal or bigger than inorganic portion. The oil-soluble compounds may be selected from oil-soluble vitamin compounds, oil-soluble terpene alcohols, phytosterol and derivatives thereof.

The amount of an oil-soluble compound may range from about 0.01% to about 10%, preferably, about 0.05% to about 5%, more preferably from about 0.1% to about 3%, by weight of the composition.

A number of vitamins known by those in the art for providing various skin benefits are oil-soluble and some or all of their derivatives are oil-soluble. Non-limiting examples of such oil-soluble vitamin compounds include retinoids, vitamin C (e.g. ascorbyl palmitate), vitamin D, vitamin K, vitamin E, and mixtures thereof. Preferred for use herein are retinoids, vitamin E, and mixtures thereof.

Oil-soluble terpene alcohols that are useful herein include farnesol, derivatives of farnesol, isomers of farnesol, geraniol, derivatives of geraniol, isomers of geraniol, phytantriol, derivatives of phytantriol, isomers of phytantriol, and mixtures thereof. Preferred for use herein is farnesol.

Phytosterol and derivatives thereof are known for providing skin lightening benefits. Non-limiting examples of oil-soluble phytosterol derivatives include β-sitosterol, campesterol, brassicasterol, lupenol, α-spinasterol, stigmasterol, their derivatives, and combinations thereof.

### Sunscreen Agents

The compositions of the subject invention may optionally contain a sunscreen agent selected from an organic sunscreen agent and an inorganic sunscreen agent.

Organic sunscreen agents useful herein include homosalate, octocrylene, octyl-p-methoxycinnamate, phenyl benzimidazole sulfonic acid, 2-hydroxy-4-methoxybenzophenone (Benzophenone-3), 2-ethylhexyl-salicylate, and mixtures thereof.

Inorganic sunscreen agents useful herein include the following metallic oxides; titanium dioxide, zinc oxide, zirconium oxide, iron oxide, and mixtures thereof.

When included in the present compositions, the sunscreens are preferably included in amounts of from about 0.1% to about 20%, preferably from about 0.5% to about 10%, more preferably from about 1% to about 5%, by weight of the composition. Exact amounts will vary depending upon the sunscreen or sunscreens chosen and the desired Sun Protection Factor (SPF).

### Thickening Agents

The compositions of the present invention, in some embodiments, may further include one or more thickening agents.

Nonlimiting classes of thickening agents include those selected from the following: carboxylic acid polymers, crosslinked polyacrylate polymers, polyacrylamide polymers, polysaccharides and gums.

When present, the composition preferably includes from about 0.01% to about 5%, more preferably from about 0.1% to about 4%, and still more preferably from about 0.1% to about 3%, by weight of the composition of the thickening agent.

### Other Optional Ingredients

A variety of additional ingredients can be incorporated into the compositions of the present invention. Nonlimiting examples of these additional ingredients include; particular materials to modify skin feel or appearance; anti-acne actives; oil-soluble beta-hydroxy acids such as salicylic acid and derivatives thereof; chelators; flavonoid compounds; anti-inflammatory agents; anti-cellulite agents; desquamation actives; anti-oxidant/radical scavengers; tanning actives; skin soothing or skin healing actives such as panthenoic acid derivatives (including panthenol, dexpanthenol, ethyl panthenol), aloe vera, allantoin, bisabolol, and dipotassium glycyrrhizinate; antimicrobial or antifungal actives.

### Composition Preparation

The compositions according to the present invention are generally prepared by conventional methods such as are known in the art of making topical compositions. Such methods typically involve mixing of the ingredients in one or more steps to a relatively uniform state, with or without heating, cooling, application of vacuum, and the like.

### Products for Topical Use

The topical compositions of the present invention may be formulated into a facial skin cosmetic, moisturizer, wrinkle soothing serum, lotion, skin facial mask, skin lotion, skin cream, skin gel, eye gel, eye cream, or any other commonly known skin product or treatment.

In one preferred embodiment, the composition of the present invention is water-in-oil emulsion.

In another preferred embodiment, the composition of the present invention has a viscosity above 10,000 cps.

### Method of Use

Applicants have found that the compositions of the present invention are useful in a variety of applications directed to enhancement of mammalian skin. The methods of use for the compositions disclosed and claimed herein include, but are not limited to: 1) methods of increasing the substantivity of a cosmetic to skin; 2) methods of moisturizing skin; 3) methods of improving the natural appearance of skin; 4) methods of applying a color cosmetic to skin; 5) methods of preventing, retarding, and/or treating wrinkles; 6) methods of providing UV protection to skin; 7) methods of preventing, retarding, and/or controlling the appearance of oil; 8) methods of modifying the feel and texture of skin; 9) methods of providing even skin tone; 10) methods of preventing, retarding, and/or treating the appear of spider vessels and varicose veins; 11) methods of masking the appearance of vellus hair on skin; and 12) methods of concealing blemishes and/or imperfections in human skin, including acne, age spots, freckles, moles, scars, under eye circles, birth marks, post-inflammatory hyperpigmentation, etc. Each of the methods discussed herein involve topical application of the claimed compositions to skin.

### Test Methods

### Viscosity Measurement

A product viscosity is measured by a commercially available viscometer like BROOKFIELD DV II+ Viscometer with Helipath T-C bar type spindle (BROOKFIELD ENGINEERING LABORATORIES, INC.) at 5 rpm/min at 25° C.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

Water in Oil emulsion skin care products are prepared by conventional methods from the following components.

TABLE 1

| | | | | | | | | | | | |
|---|---|---|---|---|---|---|---|---|---|---|---|
| (values in | | Com. | | Com. | | | Com. | | | | |
| wt %) | Ex. 1 | Ex. 1 | Ex. 2 | Ex. 2 | Ex. 3 | Ex. 4 | Ex. 4 | Ex. 5 | Ex. 6 | Ex. 7 | Ex. 8 |

Phase A

| | | | | | | | | | | | |
|---|---|---|---|---|---|---|---|---|---|---|---|
| Dimethicone *1 | 5.0 | 5.0 | 5.0 | 5.0 | 5.0 | 5.0 | 5.0 | 6.0 | 7.50 | 10.0 | — |
| SF1202 *2 | — | — | — | — | — | — | — | — | — | — | 22.0 |
| KSG-15 *3 | 5.0 | 5.0 | 5.0 | 5.0 | 5.0 | 5.0 | 5.0 | 4.0 | 7.0 | 10.0 | 15.5 |
| KSG-210 *4 | 5.0 | 5.0 | 5.0 | 5.0 | 5.0 | 5.0 | 5.0 | 6.0 | 6.0 | 7.0 | 2.5 |
| KF-6017 *5 | — | 0.75 | — | 0.75 | — | — | 0.75 | — | — | — | — |
| KF-6104 *6 | 0.75 | — | 0.75 | — | 0.75 | 0.75 | — | — | 0.5 | 0.25 | 0.75 |
| KF-6105 *7 | — | — | — | — | — | — | — | 0.75 | — | — | — |

Phase B

| | | | | | | | | | | | |
|---|---|---|---|---|---|---|---|---|---|---|---|
| Sepiwhite | 1.0 | 1.0 | 0.2 | 0.2 | 0.5 | — | — | 1.0 | 1.0 | 1.0 | 1.0 |
| MSH *8 | | | | | | | | | | | |
| Tri- | 0.45 | 0.45 | 0.09 | 0.09 | 0.23 | — | — | 0.45 | 0.45 | 0.45 | 0.45 |
| ethanolamine | | | | | | | | | | | |
| CPC *9 | — | — | — | — | — | 0.1 | 0.1 | — | — | — | — |
| Glycerin, USP | 10.0 | 10.0 | 10.0 | 10.0 | 10.0 | 10.0 | 10.0 | 10.0 | 10.0 | 10.0 | 10.0 |
| Niacinamide | 5.0 | 5.0 | 5.0 | 5.0 | 5.0 | 5.0 | 5.0 | 5.0 | 5.0 | 5.0 | 5.0 |
| PEG-32 | 2.0 | 2.0 | 2.0 | 2.0 | 2.0 | 2.0 | 2.0 | 2.0 | 2.0 | 2.0 | — |
| Pentylene | 3.0 | 3.0 | 3.0 | 3.0 | 3.0 | 3.0 | 3.0 | 3.0 | 3.0 | 3.0 | — |
| Glycol | | | | | | | | | | | |
| Sodium | 0.5 | 0.5 | 0.5 | 0.5 | 0.5 | 0.5 | 0.5 | 0.5 | 0.5 | 0.5 | — |
| Chloride | | | | | | | | | | | |
| Methyl- | 0.2 | 0.2 | 0.2 | 0.2 | 0.2 | 0.2 | 0.2 | 0.2 | 0.2 | 0.2 | 0.2 |
| paraben | | | | | | | | | | | |
| Sodium | 0.2 | 0.2 | 0.2 | 0.2 | 0.2 | 0.2 | 0.2 | 0.2 | 0.2 | 0.2 | — |
| Citrate | | | | | | | | | | | |
| Benzyl | 0.2 | 0.2 | 0.2 | 0.2 | 0.2 | 0.2 | 0.2 | 0.2 | 0.2 | 0.2 | 0.2 |
| Alcohol | | | | | | | | | | | |
| Disodium | 0.1 | 0.1 | 0.1 | 0.1 | 0.1 | 0.1 | 0.1 | 0.1 | 0.1 | 0.1 | 0.1 |
| EDTA | | | | | | | | | | | |
| Water | q.s. to 100 | q.s. to 100 | q.s. to 100 | q.s. to 100 | q.s. to 100 | q.s. to 100 | q.s. to 100 | q.s. to 100 | q.s. to 100 | q.s. to 100 | q.s. to 100 |

*1. E.g., KF96A (6cs). Available from Shin-Etsu, Tokyo, Japan.
*2. Cyclopentasiloxane. Available from Toshiba GE
*3. 5% Dimethicone/Vinyl Dimethicone Crosspolymer in Dimethicone. Available from Shin-Etsu, Tokyo, Japan.
*4. 25% Dimethicone PEG-10/15 Crosspolymer in Dimethicone. Available from Shin-Etsu, Tokyo, Japan.
*5. PEG-10 Dimethicone. Available from Shin-Etsu, Tokyo, Japan.
*6. Polyglyceryl-3 Polydimethylsiloxyethyl Dimethicone. Available from Shin-Etsu, Tokyo, Japan.
*7. Lauryl Polyglyceryl-3 Polydimethylsiloxyethyl Dimethicone. Available from Shin-Etsu, Tokyo, Japan.
*8. Undecylenoyl Phenyl Alanine. Available from Seppic.
*9. Cetylpyridinium Chloride, Available from Wako Pure Chemical Industries Ltd.

TABLE 2

| | | | | | | | | | |
|---|---|---|---|---|---|---|---|---|---|
| | | | | | | | | | Com. |
| (values in wt %) | Ex. 9 | Ex. 10 | Ex. 11 | Ex. 12 | Ex. 13 | Ex. 14 | Ex. 15 | Ex. 16 | Ex. 5 |

Phase A

| | | | | | | | | | |
|---|---|---|---|---|---|---|---|---|---|
| Dimethicone | — | — | 5.0 | 5.0 | 5.0 | 5.0 | 5.0 | 5.0 | 5.0 |
| SF1202 | 22.0 | 22.0 | — | — | — | — | — | — | — |
| KSG-15 | 15.5 | 15.5 | 5.0 | 5.0 | 5.0 | 5.0 | 5.0 | 5.0 | 5.0 |
| KSG-210 | 2.5 | 2.5 | 5.0 | 2.5 | — | — | — | 5.0 | 5.0 |
| KSG-710 *10 | — | — | — | 2.5 | 10.0 | — | — | — | — |
| KSG-810 *11 | — | — | — | — | — | 5.0 | 10.0 | — | — |
| KF-6107 | — | — | — | — | — | — | — | 0.5 | — |
| KF-6104 | 0.75 | 0.75 | 3.75 | 0.75 | — | 0.75 | — | — | 0.5 |
| KF-6105 | — | — | — | — | 0.30 | — | 0.30 | — | — |
| Octylmethoxy | — | 7.5 | — | — | — | — | — | — | — |
| cinnamate | | | | | | | | | |

Phase B

| | | | | | | | | | |
|---|---|---|---|---|---|---|---|---|---|
| Sepiwhite MSH | 1.0 | 1.0 | 5.0 | 1.0 | 1.0 | 1.0 | 1.0 | — | — |
| Olivem 460 *12 | — | — | — | — | — | — | — | 1.0 | 1.0 |
| Triethanolamine | 0.45 | 0.45 | 2.25 | 0.45 | 0.45 | 0.45 | 0.45 | — | — |
| Glycerin, USP | 20.0 | 10.0 | 10.0 | 10.0 | 10.0 | 10.0 | 10.0 | 10.0 | 10.0 |
| Niacinamide | 5.0 | 5.0 | 5.0 | 5.0 | 5.0 | 5.0 | 5.0 | 5.0 | 5.0 |
| PEG-32 | — | — | 2.0 | 2.0 | 2.0 | 2.0 | 2.0 | 2.0 | 2.0 |
| Pentylene Glycol | — | — | 3.0 | 3.0 | 3.0 | 3.0 | 3.0 | 3.0 | 3.0 |
| Sodium Chloride | — | — | 0.5 | 0.5 | 0.5 | 0.5 | 0.5 | 0.5 | 0.5 |
| Methylparaben | 0.2 | 0.2 | 0.2 | 0.2 | 0.2 | 0.2 | 0.2 | 0.2 | 0.2 |
| Sodium Citrate | — | — | 0.2 | 0.2 | 0.2 | 0.2 | 0.2 | 0.2 | 0.2 |
| Benzyl Alcohol | 0.2 | 0.2 | 0.2 | 0.2 | 0.2 | 0.2 | 0.2 | 0.2 | 0.2 |
| Disodium EDTA | 0.1 | 0.1 | 0.1 | 0.1 | 0.1 | 0.1 | 0.1 | 0.1 | 0.1 |
| Zinc oxide *13 | — | 3.0 | — | — | — | — | — | — | — |
| Water | q.s. to 100 | q.s. to 100 | q.s. to 100 | q.s. to 100 | q.s. to 100 | q.s. to 100 | q.s. to 100 | q.s. to 100 | q.s. to 100 |

*10. Dimethicone/Polyglycerin-3 Crosspolymer and Dimethicone. Available from Shin-Etsu, Tokyo, Japan.
*11. Lauryl Dimethicone/Polyglycerin-3 crosspolymer and Mineral oil. Available from Shin-Etsu, Tokyo, Japan.
*12. Sodium PEG-7 Olive Oil Caroboxylate & water: Available from B&T SRL
*13. Zinc Oxide, Z-Cote HP1: available from BASF Corp.

In separate suitable containers are added the ingredients of Phase A and Phase B and each phase is mixed using a suitable mixer (e.g., Anchor blade, propeller blade, IKA T25). When each phase is homogenous, slowly add Phase B to Phase A while mixing Phase A with a suitable mixer (e.g., Anchor blade, propeller blade, IKA T25). Maintain mixing until batch is uniform. Pour product into suitable containers.

Viscosities of selected examples and comparative examples were measured 24 hours, and 2 weeks or 1 month after completion of formulating each composition according to the VISCOSITY MEASURMENT, and are summarized the table below:

TABLE 3

| | | Com. | | Com. | | Com. |
|---|---|---|---|---|---|---|
| | Ex. 1 | Ex. 1 | Ex. 2 | Ex. 2 | Ex. 4 | Ex. 4 |
| Viscosity after 24 hr | 35,760 | Phase | 35,680 | 4,640 | 12,400 | 10,960 |
| at 25° C. (cps) | | separation | | | | |
| | | observed | | | | |
| Viscosity after | 31,360 | Phase | 36,640 | Phase | 12,200 | Phase |
| 1 month at 25° C. | | separation | | separation | | separation |
| (cps) | | observed | | observed | | observed |

TABLE 4

| | Ex. 16 | Com. Ex. 5 |
|---|---|---|
| Viscosity after 24 hr | 17,346 | 20 |
| at 25° C. (cps) | | |
| Viscosity after 2 weeks | 12,347 | Phase separation observed |
| at 25° C. (cps) | | at day 2 |

It is understood that the foregoing detailed description of examples and embodiments of the present invention are given merely by way of illustration, and that numerous modifications and variations may become apparent to those skilled in the art without departing from the spirit and scope of the invention; and such apparent modifications and variations are to be included in the scope of the appended claims.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic composition comprising:
a) from about 0.1% to about 15% of an emulsifying crosslinked siloxane elastomer;
b) from about 0.1% to about 10% of a noncrosslinked silicone emulsifier having a polyglycerin unit;
c) from about 1% to about 50% of a solvent for the emulsifying crosslinked siloxane elastomer;
d) from about 0.05% to about 10% of an amphiphilic active; and
e) water.

2. A composition according to claim 1, wherein said emulsifying crosslinked siloxane elastomer is a dimethicone copolyol crosspolymer.

3. A composition according to claim 1, wherein said noncrosslinked silicone emulsifier having a polyglycerin unit is a dimethicone copolyol.

4. A composition according to claim 1, wherein said solvent for the emulsifying crosslinked siloxane elastomer is selected from the group consisting of volatile, non-polar oils; non-volatile, polar oils; non-volatile, non-polar oils; non-volatile paraffinic hydrocarbon oils; and mixtures thereof.

5. A cosmetic composition according to claim 4, wherein said solvent for the emulsifying crosslinked siloxane elastomer is silicone oil.

6. A cosmetic composition according to claim 1, wherein said amphiphilic active is selected from a group consisting of undecenoyl phenylalanine, cetylpyridinium chloride, a glycyrrhizate salt, sodium dilauramidoglutamide lysine, sodium PEG-7 olive oil carboxylate and mixtures thereof.

7. A cosmetic composition according to claim 6, wherein said amphiphilic active is undecenoyl phenylalanine.

8. A cosmetic composition according to claim 6, wherein said amphiphilic active is cetylpyridinium chloride.

9. A cosmetic composition according to claim 6, wherein said amphiphilic active is sodium PEG-7 olive oil carboxylate.

10. A cosmetic composition according to claim 1, wherein said composition further comprises from about 0.1% to about 15% of a non-emulsifying crosslinked siloxane elastomer.

11. A composition according to claim 1, wherein said composition further comprises a skin care active selected from the group consisting of vitamin B₃ compounds, whitening agents, peptides, sugar amines, and mixtures thereof.

12. A composition according to claim 1, wherein said composition further comprises sunscreen agents.

13. A cosmetic composition according to claim 1, wherein said composition has a viscosity above 10,000 cps.

14. A cosmetic composition according to claim 1, wherein said composition is water-in-oil emulsion.

15. A cosmetic composition according to claim 14, wherein said composition contains the amphiphilic active in an aqueous phase.

16. A method of regulating the condition of skin, said method comprises applying to the skin of a human, a safe and effective amount of a composition according to claim 1.
